(19) United States
(12) Patent Application Publication
(10) Pub. No.: US 2026/0078339 A1
(43) Pub. Date: Mar. 19, 2026

(54) HLA Triple Knockout Human Follicle Cells for Enhanced Hair Implantation Therapy with Integrated Biosensor System

(71) Applicant: Reem Mahrat, Pleasanton, CA (US)

(72) Inventor: Reem Mahrat, Pleasanton, CA (US)

(21) Appl. No.: 18/887,146

(22) Filed: Sep. 17, 2024

(51) Int. Cl. C12N 5/071; A61F 2/10; C12N 9/22; C12N 15/11; C12N 15/90

(52) U.S. Cl. CPC C12N 5/0628; C12N 9/22; C12N 15/11; C12N 15/907; A61F 2/10; C12N 2310/20

(57) ABSTRACT

The present invention discloses a novel method for developing Human HLA-a/b/c triple knockout hair follicle stem cells (HFSCs) intended for promoting HFSCS transplantation. The method involves precise genetic modification techniques to knockout HLA-a/b/c genes, facilitating enhanced compatibility and effectiveness in hair follicles transplantation. The inventive approach aims to address challenges (donor dependent and large scar) in hair implantation therapy by leveraging advanced gene-editing and stem cell technologies.

### FIELD OF INVENTION

The present invention is directed towards engineered cells containing one or more modifications, such as genetic modifications, for allogeneic cell therapy.

### BACKGROUND

Hair loss and alopecia present significant challenges worldwide, impacting individuals' self-esteem and quality of life. There are many treatments available to cure hair loss, including but not limited to medications, hair transplant, platelet rich plasma(PRP). Hair transplant is a surgery in which the hair is moved from thick areas to bald areas of the scalp. Nowadays stem cell therapy is also used to treat alopecia. In this technique, stem cells are used to control hair loss. Firstly, the healthy follicles are extracted from the scalp and processed in the laboratory, secondly, these follicles are inserted to the bald patches of the scalp using microneedles.

Conventional hair implantation treatments have two major challenges: donor dependent and large scar, prompting the exploration of innovative approaches, including stem cell-based therapies. Generating HLA triple knockout hair follicle cells is an attractive solution to solve the above two major challenges in HFSCs implantation therapy and ensure success.

Human Leukocyte Antigen (HLA-a/b/c) plays a critical role in identifying and attacking foreign cells, which are the major hurdles for the survival of transplanted tissue/organs. Compromising HLA-a/b/c function will significantly increase the compatibility of transplanted tissue/organ with recipients.

### SUMMARY

The present invention discloses a novel method for developing Human HLA-a/b/c triple knockout hair follicle stem cells (HFSCs) intended for promoting HFSCS transplantation. The method involves precise genetic modification techniques to knockout HLA-a/b/c genes, facilitating enhanced compatibility and effectiveness in hair follicles transplantation. The inventive approach aims to address challenges (donor dependent and large scar) in hair implantation therapy by leveraging advanced gene-editing and stem cell technologies.

### DESCRIPTION

The present invention now will be described hereinafter with reference to the accompanying examples and/or drawings, in which embodiments of the invention are shown. This description is not intended to be a detailed catalog of all the different ways in which the invention may be implemented or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

The present invention pertains to the field of regenerative medicine, focusing on the development of immuno-tolerant hair follicle stem cells (HFSCs) for hair implantation therapy. The invention involves sophisticated gene-editing techniques to knockout Human Leukocyte Antigen (HLA-a/b/c) genes in HFSCs, thereby enhancing the compatibility and effectiveness of hair follicle transplantation. Hair loss and alopecia significantly impact individuals' self-esteem and quality of life. Current hair implantation therapies face two major challenges: dependency on donor hair and the creation of large scars. These limitations necessitate innovative approaches, such as stem cell-based therapies. The generation of HLA triple knockout hair follicle cells presents a promising solution to these challenges, ensuring successful HFSC implantation therapy.

The present invention describes a novel method for creating HLA-a/b/c triple knockout HFSCs, aimed at enhancing the compatibility and effectiveness of hair follicle transplantation. This method employs precise genetic modification techniques using CRISPR/Cas9 technology, combined with a biosensor system to ensure the quality and stemness of the HFSCs. This approach aims to address issues related to donor dependency and large scars in hair implantation therapy. The method for developing HLA-a/b/c triple knockout HFSCs with a biosensor system involves several key steps. Initially, HFSCs are isolated and cultured, characterized by the expression of CD105 and the absence of CD45 surface markers. Subsequently, a biosensor system is established by creating an HFSC cell line with CD105 linked to GFP (green fluorescence) and CD45 linked to RFP (red fluorescence) using the CRISPR/Cas9 knock-in method. In this system, undifferentiated HFSCs express green fluorescence, while differentiated follicle cells express red fluorescence. Fluorescence-activated cell sorting (FACS) is used to select green fluorescent cells, ensuring the expansion of undifferentiated HFSCs. These selected green HFSCs are then expanded, maintaining the biosensor system to ensure the integrity and purity of the cells. Next, HLA-a/b/c triple knockout is established by designing and synthesizing guide RNAs (gRNAs) targeting HLA-a/b/c genes for precise gene modification using CRISPR/Cas9 technology. The HFSCs with the biosensor system are transfected with the designed gRNAs, and HLA-a/b/c triple knockout cells are selected using genomic sequencing. Single-clone HLA-a/b/c triple knockout HFSCs with the biosensor system are then expanded. Quality control of the stemness of HLA-a/b/c triple knockout HFSCs is ensured using the biosensor system. Only green fluorescent cells are expanded, as FACS removes differentiated cells (red fluorescent). The hair follicle growth potential of these cells is evaluated both in vitro and in vivo. In vitro, HLA-a/b/c triple knockout HFSCs with the biosensor system can be differentiated into osteocytes and adipocytes. In vivo, their growth potential is evaluated using a mouse model. Finally, protocols for the large-scale production of HLA triple knockout HFSCs with the biosensor system are developed and optimized. This method offers several benefits, including improved immune compatibility by eliminating immuno-rejection, enhanced efficacy by reducing dependence on donor hair for implantation, customizable approaches through targeted gene editing for personalized therapies, and scalable production protocols for widespread application. The present invention as described above shall be described in detail below.

Embodiments of the invention are associated with various advantages and/or technical effects. The present application relates to hair follicle stem cells and uses thereof, and more particularly to methods and compositions for isolating, culturing, and utilizing hair follicle stem cells for various applications. This application addresses the problem of producing immuno-tolerant hair follicle stem cells (HFSCs) for potential use in hair follicle regeneration therapies. The application provides solutions for isolating and culturing HFSCs, genetically modifying them to create HLA-a/b/c triple knockout HFSCs, and establishing a biosensor system to select undifferentiated HFSCs. The application also addresses the need for expanding and confirming the genetic modification of the HFSCs, as well as evaluating their hair follicle growth potential in vitro and in vivo.

One embodiment of the present invention provides a method for producing immuno-tolerant hair follicle stem cells (HFSCs), comprising the steps of isolating and culturing HFSCs, genetically modifying the HFSCs to create HLA-a/b/c triple knockout HFSCs. The creation of HLA-a/b/c triple knockout HFSCs significantly reduces the risk of immune rejection when these cells are used for hair transplantation therapies, thereby increasing the success rate of such treatments. The genetic modification of HFSCs to remove HLA antigens provides a universal donor cell line, potentially making these cells suitable for a wide range of patients without the need for individual HLA matching. The method provides the advantage of producing immuno-tolerant hair follicle stem cells (HFSCs) by genetically modifying the cells to create HLA-a/b/c triple knockout HFSCs, thereby reducing the risk of immune rejection in transplantation procedures.

Another embodiment of the invention provides the establishment of a biosensor system to select undifferentiated HFSCs, which allows for the identification and isolation of the most suitable cells for further expansion and genetic modification. The method utilizes a biosensor system comprising CD105 linked GFP and CD45 linked RFP, enabling the selective identification of undifferentiated HFSCs based on green fluorescence emission. This facilitates the efficient and accurate selection of the desired cells.

According to another embodiment, the method incorporates the use of CRISPR/Cas9 technology for genetic modification, which offers a precise and efficient tool for creating the HLA-a/b/c triple knockout in HFSCs. This ensures the reliability and standardization of the genetic modification process.

According to an embodiment, the method includes the design and synthesis of gRNAs for HLA-a/b/c genes, allowing for targeted and specific genetic modification. This enhances the efficiency and effectiveness of the genetic modification process, leading to a higher success rate in creating the desired HLA-a/b/c triple knockout HFSCs.

In a development, the method further comprises establishing a biosensor system to select undifferentiated HFSCs. The establishment of a biosensor system for selecting undifferentiated HFSCs ensures the purity of the stem cell population, which is critical for maintaining their regenerative capabilities and preventing differentiation prior to therapeutic use. The biosensor-based selection process enhances the efficiency of isolating the desired stem cell population, reducing the time and resources required for cell sorting and increasing the overall yield of viable HFSCs for clinical applications. In a development, the method further comprises a biosensor system comprising a CD105 linked GFP and a CD45 linked RFP. The use of a CD105 linked GFP and a CD45 linked RFP in the biosensor system allows for the simultaneous detection of stem cell markers and the exclusion of unwanted cell types, such as hematopoietic cells, thereby improving the specificity of the selection process.

According to an embodiment of the present invention the dual-fluorescence biosensor system enables real-time monitoring of cell populations during the selection process, facilitating rapid adjustments to culturing conditions and improving the overall quality of the stem cell product.

According to an embodiment, the method further comprises undifferentiated HFSCs being selected based on green fluorescence emission. Selecting undifferentiated HFSCs based on green fluorescence emission streamlines the identification of target cells, allowing for a more rapid and straightforward selection process compared to conventional methods that may require multiple steps or additional reagents. The reliance on green fluorescence for cell selection minimizes the potential for cell damage or alteration that can occur with more invasive selection techniques, preserving the integrity and therapeutic potential of the HFSCs.

In another embodiment, the method further comprises expanding the selected undifferentiated HFSCs. Expanding the selected undifferentiated HFSCs ensures an ample supply of cells for therapeutic applications, enabling the treatment of larger areas or multiple patients from a single isolation and selection procedure. The expansion of a pure population of undifferentiated HFSCs maintains the consistency and predictability of the cell product, which is essential for regulatory approval and clinical reliability.

In yet another embodiment, the method further comprises a genetic modification being performed using CRISPR/Cas9 technology. The use of CRISPR/Cas9 technology for genetic modification provides a high degree of precision in editing the target genes, reducing off-target effects and increasing the specificity of the genetic alteration. The application of CRISPR/Cas9 allows for the introduction of targeted modifications at a reduced cost and with greater efficiency compared to traditional gene-editing methods, thereby accelerating the development process of genetically modified cells.

The method further comprises designing and synthesizing gRNAs for HLA-a/b/c genes. Designing and synthesizing gRNAs for HLA-a/b/c genes enables targeted gene editing, allowing for the precise knockout of specific alleles that may be implicated in immune rejection during cell transplantation. The ability to create gRNAs specific to HLA-a/b/c genes facilitates the study of these genes' functions in immune recognition and may lead to the development of novel therapies for autoimmune diseases and transplant rejection. In development, the method further comprises an HLA-a/b/c triple knockout being confirmed by genomic sequencing. Confirming an HLA-a/b/c triple knockout by genomic sequencing ensures the complete elimination of the targeted genes, which is critical for reducing the risk of immune rejection in therapeutic applications. Genomic sequencing verification provides a reliable method for quality control, ensuring that the cell line modifications meet the necessary criteria for subsequent experimental and clinical use.

According to an embodiment, the method further comprises expanding HLA-a/b/c triple knockout single-clone HFSCs. Expanding HLA-a/b/c triple knockout single-clone HFSCs allows for the generation of a homogeneous cell population, which is essential for consistent experimental outcomes and potential therapeutic applications. The expansion of single-clone cells ensures the maintenance of the genetic and phenotypic characteristics of the original knockout cell, providing a stable platform for further research and development.

In yet another embodiment, the method further comprises evaluating the hair follicle growth potential of the HLA-a/b/c triple knockout HFSCs in vitro and in vivo. Evaluating the hair follicle growth potential of the HLA-a/b/c triple knockout HFSCs in vitro provides a controlled environment to assess the functional impact of the genetic modifications on hair follicle development and regeneration. In vivo assessment of hair follicle growth potential confirms the translational relevance of the in vitro findings and provides critical data on the safety and efficacy of the genetically modified HFSCs when used in potential therapeutic contexts.

A further embodiment of the present invention provides a system for producing immuno-tolerant hair follicle stem cells (HFSCs), comprising the steps of a culture medium for HFSCs, a CRISPR/Cas9 gene-editing toolkit for creating HLA-a/b/c triple knockout HFSCs, a biosensor system for selecting undifferentiated HFSCs. The culture medium specifically designed for HFSCs provides an optimized environment that promotes the growth and maintenance of hair follicle stem cells, which can enhance the efficiency and stability of the cell culture process. Utilizing the CRISPR/Cas9 gene-editing toolkit for creating HLA-a/b/c triple knockout HFSCs allows for the precise and targeted modification of genomic DNA, potentially leading to the generation of immuno-tolerant cells that can be used in therapeutic applications without eliciting an immune response. The inclusion of a biosensor system for selecting undifferentiated HFSCs ensures the purity of the stem cell population, which is crucial for maintaining their pluripotency and for the success of subsequent applications in regenerative medicine. The system further comprises a biosensor system including a CD105 linked GFP and a CD45 linked RFP. The biosensor system with CD105 linked GFP and CD45 linked RFP provides a dual-color fluorescence approach that enables the simultaneous identification and isolation of HFSCs based on specific surface markers, thereby increasing the accuracy of cell sorting. The use of fluorescent proteins linked to cell surface markers allows for real-time monitoring of HFSCs, facilitating the rapid detection of changes in the cell population and ensuring the selection of the most suitable cells for downstream applications. The system further comprises a biosensor system being configured to facilitate fluorescence-activated cell sorting (FACS) based on green fluorescence emission. The biosensor system configured to facilitate FACS based on green fluorescence emission allows for high-throughput sorting of cells, significantly speeding up the process of isolating the desired HFSCs from a heterogeneous cell mixture. The ability to sort cells using FACS based on green fluorescence emission enhances the purity of the selected HFSCs, which is critical for ensuring the quality and reproducibility of experimental results and potential clinical outcomes.

The system further comprises a genomic sequencing setup to confirm the HLA-a/b/c triple knockout in HFSCs. The genomic sequencing setup to confirm the HLA-a/b/c triple knockout in HFSCs provides a definitive method for verifying the successful genetic modification of the cells, which is essential for ensuring that the HFSCs are indeed immuno-tolerant. The use of genomic sequencing allows for the detection of off-target effects that may arise from the CRISPR/Cas9 gene-editing process, thereby ensuring the safety and reliability of the modified HFSCs for therapeutic use.

The system further comprises a genomic sequencing setup being adapted to validate the expansion of HLA-a/b/c triple knockout single-clone HFSCs. The genomic sequencing setup adapted to validate the expansion of HLA-a/b/c triple knockout single-clone HFSCs ensures that the cell line remains genetically stable throughout the expansion process, which is vital for maintaining the immuno-tolerant properties of the cells. By confirming the genetic integrity of expanded single-clone HFSCs, the sequencing setup provides a quality control measure that supports the scalability of cell production for larger-scale applications, such as tissue engineering and transplantation.

The system further comprises an in vitro assay setup for evaluating hair follicle growth potential. The inclusion of an in vitro assay setup enables the rapid screening of compounds or treatments for their effects on hair follicle growth, thereby reducing the time and resources required for preliminary investigations. The system allows for controlled experimental conditions, minimizing external variables that could affect the reproducibility and reliability of the results. Utilizing an in vitro assay setup facilitates the assessment of hair follicle growth potential at a cellular level, providing detailed insights into the mechanisms of action of the tested compounds or treatments. The system further comprises an in vitro assay setup being complemented by an in vivo model for further validation of hair follicle growth potential. The combination of in vitro and in vivo models provides a comprehensive evaluation platform, ensuring that findings are not only experimentally sound but also applicable to living organisms. An in vivo model complements the in vitro assay by validating the physiological relevance of the observed effects, enhancing the predictive value of the system for clinical outcomes. The use of an in vivo model allows for the assessment of systemic factors and long-term effects on hair follicle growth, which cannot be fully replicated in an in vitro environment.

In conclusion, the development of HLA triple knockout HFSCs with a biosensor system represents a significant advancement in hair implantation therapy. This innovative method addresses key challenges in hair restoration, such as donor dependency and scarring, by leveraging advanced gene-editing and stem cell technologies. The approach outlined in this patent application holds immense potential for revolutionizing hair restoration therapies and improving patients'quality of life.

Because many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

1. A method for producing immuno-tolerant hair follicle stem cells (HFSCs), comprising:
a. isolating and culturing HFSCs;
b. genetically modifying the HFSCs to create HLA-a/b/c triple knockout HFSCs;
c. a biosensor system established to select undifferentiated HFSCs, wherein the biosensor system comprises a CD105 linked GFP and a CD45 linked RFP.

2. The method of claim 1, wherein undifferentiated HFSCs are selected based on green fluorescence emission.

3. The method of claim 1, further comprises expanding the selected undifferentiated HFSCs.

4. The method of claim 1, wherein the genetic modification is performed using CRISPR/Cas9 technology.

5. The method of claim 1, further comprises designing and synthesizing gRNAs for HLA-a/b/c genes.

6. The method of claim 1, further comprising expanding HLA-a/b/c triple knockout single-clone HFSCs.

7. A system for producing immuno-tolerant hair follicle stem cells (HFSCs), comprising: a. a culture medium for HFSCs; b. a CRISPR/Cas9 gene-editing toolkit for creating HLA-a/b/c triple knockout HFSCs; c. a biosensor system for selecting undifferentiated HFSCs.

8. The system of claim 7, wherein the biosensor system includes a CD105 linked GFP and a CD45 linked RFP.

9. The system of claim 7, wherein the biosensor system is configured to facilitate fluorescence-activated cell sorting (FACS) based on green fluorescence emission.

10. The system of claim 7, further comprising a genomic sequencing setup to confirm the HLA-a/b/c triple knockout in HFSCs.

11. The system of claim 7, wherein the genomic sequencing setup is adapted to validate the expansion of HLA-a/b/c triple knockout single-clone HFSCs.
